(19) United States
(12) Patent Application Publication
(10) Pub. No.: US 2022/0354960 A1
(43) Pub. Date: Nov. 10, 2022

(54) IMMUNE CHECKPOINT BLOCKING BISPECIFIC MOLECULES

(71) Applicant: THE SCRIPPS RESEARCH INSTITUTE, La Jolla, CA (US)

(72) Inventors: Peter Schultz, La Jolla, CA (US); Feng Wang, Carlsbad, CA (US); Ray Changming Fang, San Diego, CA (US); Yu J. Cao, Shenzhen, Guangdong (CN); Weijun Shen, San Diego, CA (US); Xiaoxuan Lyu, San Diego, CA (US); Sam Lear, La Jolla, CA (US)

(21) Appl. No.: 17/621,410

(22) PCT Filed: Jun. 12, 2020
(86) PCT No.: PCT/US2020/037518

§ 371 Date: Dec. 21, 2021

Related U.S. Application Data
(60) Provisional application No. 62/861,524, filed on Jun. 14, 2019.

(51) Int. Cl. A61K 47/68; C07K 14/575; C07K 16/28; A61P 35/00

(52) U.S. Cl. CPC A61K 47/6849; A61K 47/6811; C07K 14/575; C07K 16/2818; A61P 35/00

(57) ABSTRACT

The invention provides tumor targeting, immune checkpoint-blocking bispecific molecules. The bispecific molecules contain an anti-PD-L1 antibody or antigen-binding fragment, and a peptide agent that specifically binds to a surface antigen or cellular marker of a solid tumor. Also provided in the invention are methods of using such specific molecules in various therapeutic applications.

### CROSS-REFERENCE TO RELATED APPLICATIONS

The subject patent application claims the benefit of priority to U.S. Provisional Patent Application No. 62/861,524 (filed Jun. 14, 2019). The full disclosure of the priority application is incorporated herein by reference in its entirety and for all purposes.

### BACKGROUND OF THE INVENTION

A major focus of cancer drug development is the generation of therapeutics that block immune escape by cancer cells. To date, several antibodies modulating immune checkpoints have been approved as drugs. For example, the anti-cytotoxic T-lymphocyte antigen-4 (CTLA-4) antibody, Ipilimumab, was approved for the treatment of melanoma in 2011, and the anti-programmed cell death-1 (PD-1) antibodies Nivolumab and Pembrolizumab were approved for advanced melanoma and non-small cell lung cancer (NSCLC) in 2014, respectively. The clinical efficacy of these antibodies is impressive, in which Ipilimumab and Pembrolizumab have raised the three-year survival of patients with melanoma to ˜70%, and overall survival (>5 years) to ˜30%.

However, the success of these therapies is somewhat dampened by the lack of response in many patients. For example, in advanced-stage NSCLC and SCLC, only 15%-20% of patients treated with PD-1 or PD-L1 targeted antibodies have effective and durable responses. To overcome these drawbacks, several approaches have been pursued including combining two different immune checkpoint blocking antibodies to increase response rates, and combining immunotherapy with chemotherapy or radiotherapy to enhance clinical efficacy. However, current immune checkpoint inhibitors are not tumor-specific, and induce systemic immune activation in other tissues and organs. Combination immunotherapies further amplify these toxicities, e.g., treatment with a combination of Ipilimumab and Nivolumab increased the occurrence of severe side effects by 2-4 fold compared to the monotherapies alone.

There is a need in the art for better and more effective immune checkpoint targeting drugs for cancer therapy. The instant invention is directed to addressing these and other needs.

### SUMMARY OF THE INVENTION

In one aspect, the invention provides bispecific molecules that contain a PD-L1 antibody or antigen-binding fragment thereof, and at least one peptide agent that specifically binds to an antigen or molecular marker on the surface of a tumor cell. In some of these bispecific molecules, the PD-L1 antibody is monoclonal antibody Avelumab, Durvalumab, or Atezolizumab. In various embodiments, the bispecific molecules are intended to target melanoma cells, breast cancer cells, lung cancer cells, kidney cancer cells, esophageal cancer cells, gastrointestinal cancer cells or pancreatic cancer call. In some embodiments, the tumor cell to be targeted by the bispecific molecule is melanoma cell, and the peptide agent in the bispecific molecule specifically binds to MC1R. In some of these embodiments, the peptide agent is α-MSH an analog thereof, or a variant thereof. In some of these embodiments, the employed peptide agent is NDP-MSH or a conservatively modified variant thereof.

In some embodiments, the PD-L1 antibody or antigen-binding fragment thereof is covalently fused to the peptide agent in the bispecific molecule of the invention. In some embodiments, the peptide agent is fused to the constant region of a heavy chain or a light chain of the antibody. In some embodiments, the peptide agent is fused to the N-terminus of a heavy chain or a light chain of the antibody. In some of these embodiments, the peptide agent is fused to the antibody or antigen-binding fragment thereof via an engineered N-terminal residue on the antibody (e.g., an engineered Ser residue). In some of these embodiments, the peptide agent is fused to the antibody or antigen-binding fragment thereof via a linker. In some embodiments, the employed linker is a PEG linker. In some embodiments, the employed linker is a peptide linker. In some embodiments, each antibody molecule is fused to about 1 to 10 molecules of the peptide agent in the bispecific molecule of the invention.

In another aspect, the invention provides pharmaceutical compositions that contain a therapeutically effective amount of the bispecific molecule described herein and a pharmaceutically acceptable carrier.

In another aspect, the invention provides methods for treating a solid tumor in a subject. These methods entail administering to the subject a pharmaceutical composition harboring a bispecific molecule that is comprised of a PD-L1 antibody or antigen-binding fragment thereof and a peptide agent that specifically binds to a cell surface antigen or molecular marker of the tumor. In some embodiments, the administered bispecific molecule contains a PD-L1 antibody Avelumab, Durvalumab, or Atezolizumab. Some of the methods are directed to treating melanoma. In some of these embodiments, the cell surface antigen targeted by the bispecific molecule is MC1R, and the peptide agent in the bispecific molecule is α-MSH, an analog thereof or a variant thereof. In some methods, the peptide agent is covalently linked to a heavy chain constant region or a heavy chain N-terminus of the antibody via a linker sequence in the bispecific molecule. In some of these embodiments, the employed linker is a PEG linker. In some embodiments, the employed linker is a peptide linker.

A further understanding of the nature and advantages of the present invention may be realized by reference to the remaining portions of the specification and claims.

### DESCRIPTION OF THE DRAWINGS

FIG. 1 shows the scheme of synthesis of NDP-MSH-αPD-L1 antibody-peptide conjugates. Structures of the linker for functionalizing the antibody (NHS-BCN) and the PEG derivatized peptide agent NDP-MSH are shown. Amino acid sequence of the NDP-MSH peptide is also shown (SEQ ID NO:1).

FIG. 2 shows characterization of anti-PD-L1 antibody and antibody conjugates. (A) Characterization of anti-PD-L1 antibody, NDP-MSH-αPD-L1 and NR-αPD-L1 conjugates with SDS-PAGE. Proteins were loaded with or without 50 uM DTT reduction. (B) The overall ligand-antibody ratio (LAR) for the MSH-αPD-L1 conjugate was 3.5. The distribution of the conjugation sites of NDP-MSH-αPD-L1 was determined by mass spectrometry. (C) ESI-MS analysis the molecular weight distribution of NDP-MSH-αPD-L1 and NR-αPD-L1 conjugates. The N-glycans were removed by incubation with PNGase F (Promega, PBS pH 7.4, 37° C., and 12 hr).

FIG. 3 shows in vitro activities of NDP-MSH-αPD-L1 conjugates. (A) Binding of NDP-MSH-αPD-L1, NR-αPD-L1, and αPD-L1 to Fc-fused human PD-L1 extracellular domain was detected by HRP-labeled polyclonal anti-human kappa light chain antibody using an ELISA. Error bars represent SD of triplicate samples. (B) NDP-MSH-αPD-L1 conjugates bound to the cell surface of HEK293-MC1R (MC1R⁺/PD-L1⁻) cells in a cell surface ELISA in a dose dependent fashion. (C) The binding of NDP-MSH-αPD-L1 (30 nM) to HEK293-MC1R cells was competed by free MSH peptide dose-dependently. αPD-L1 is denoted “Ave” in the figure.

FIG. 4 shows pharmacokinetics and in vivo efficacy of NDP-MSH-αPD-L1. (A) Pharmacokinetics of NDP-MSH-αPD-L1 and controls in mouse. NDP-MSH-αPD-L1 in PBS or controls was injected intraperitoneally into mice at 4 mg/kg (n=3/group), and serum was isolated for determination of conjugate concentration. Concentration vs. time curves were evaluated by non-compartmental analysis using WinNonlin. Values shown are averages of three rats in the group. t_{1/2}, half-life; tₘₐₓ, maximum concentration time; Cₘₐₓ, maximum concentration; AUC_{0-inf}, area under the concentration-time curve extrapolated to infinity. (B) In vivo efficacy of NDP-MSH-αPD-L1 in mouse B16-SIY melanoma syngeneic models (n=10/group). Tumor was measured three times a week with calipers and tumor volume was calculated. Each data point represents mean tumor volume of ten mice in each group±SD. Arrows indicate the time of drug injection. p values <0.05 compared to the control groups (saline) were considered significant. αPD-L1 is denoted “Ave” in the figure.

FIG. 5 shows results of LC-MS analysis of NDP-MSH-anti-PD-L1 heavy chain N-terminal conjugates.

FIG. 6 shows structures of α-MSH analog peptides with different linkers and conjugation sites for N-terminal attachment to PD-L1 antibody Atezolizumab.

FIG. 7 shows results from studies to characterize binding activities of the conjugates to the human PD-L1 and MC1R targets.

FIG. 8 shows results from pharmacokinetic (PK) study in mice to determine the serum half-life and exposure of NDP-MSH-αPD-L1. (A) MC1R functional assay was used to calculate the NDP-MSH exposure in plasma; and (B) ELISA based assays was used to quantify the plasma concentration of PD-L1 and Synagis backbone.

FIG. 9 shows results from in vivo efficacy studies of NDP-MSH-αPD-L1 in mouse B16-SIY melanoma syngeneic model. 1 million B16-SIY cells in 100 μL PBS was injected subcutaneously into C57/B6 mice (n=8) at day 0, and vehicle, NDP-MSH-anti-PD-L1, NDP-MSH-Synagis and PD-L1 antibody was dosed I.P. at 4MPK at day 5, 8, 11 and 14. Tumor size was measured 3×/week. Excellent anti-tumor efficacy was observed with NDP-MSH-anti-PD-L1, tumor free animals at day 26 (6 out of 8) and at day 45 (2 out of 8).

### DETAILED DESCRIPTION

### I. Overview

The invention is predicated in part on the studies undertaken by the inventors on introduction of a tumor-specific targeting element into immune checkpoint blockers, with a goal to decrease damage to normal tissues caused by systemic immune responses. This approach should result in an improved therapeutic index and facilitating combination checkpoint therapies. As detailed herein, the inventors synthesized bispecific antibodies NDP-MSH-αPD-L1 by conjugating an MSH analog to the anti-PD-L1 (αPD-L1) antibody Avelumab or Atezolizumab. MSH specifically targets the MC1R receptor on melanocytes. It was observed that the exemplified bispecific antibodies retain binding affinity for both MC1R and PD-L1, and displayed similar thermal stability, serum stability, and PK properties to that of its parental αPD-L1 antibody. The inventors also examined efficacy of the bispecific molecule in an established B16-SIY melanoma syngeneic mice model, and found that the exemplified antibodies are more efficacious than either αPD-L1 antibody or the NR-αPD-L1. In addition, tumor-infiltrating lymphocytes (TILs) analysis revealed an increase in the number of infiltrated T cells. Together, these studies demonstrate that the incorporation of a targeting element into an immune checkpoint blocking antibody can enhance antitumor activity relative to anti-immune checkpoint therapy alone, and that the bispecific molecules exemplified herein could facilitate therapies using a combination of checkpoint antibodies.

The invention accordingly provides immune checkpoint blocking bispecific molecules that contain a PD-L1 antibody, or antibody-based binding protein or antigen-binding fragment derived therefrom, and at least one peptide or polypeptide agent, that specifically binds to an antigen or molecular marker on the surface of a tumor cell. The invention also provides therapeutic applications of the immune checkpoint blocking bispecific molecules described herein in treating or preventing various solid tumors.

The following sections provide more detailed guidance for making and using the compositions of the invention, and for carrying out the methods of the invention.

### II. Definitions

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). In addition, the following definitions are provided to assist the reader in the practice of the invention.

The term “antibody” also synonymously called “immunoglobulins” (Ig), or “antigen-binding fragment” refers to polypeptide chain(s) which exhibit a strong monovalent, bivalent or polyvalent binding to a given antigen, epitope or epitopes. Unless otherwise noted, antibodies or antigen-binding fragments used in the invention can have sequences derived from any vertebrate species. They can be generated using any suitable technology, e.g., hybridoma technology, ribosome display, phage display, gene shuffling libraries, semi-synthetic or fully synthetic libraries or combinations thereof. Unless otherwise noted, the term “antibody” as used in the present invention includes intact antibodies, antigen-binding polypeptide fragments and other designer antibodies that are described below or well known in the art (see, e.g., Serafini, J Nucl. Med. 34:533-6, 1993).

An intact “antibody” typically comprises at least two heavy (H) chains (about 50-70 kD) and two light (L) chains (about 25 kD) inter-connected by disulfide bonds. The recognized immunoglobulin genes encoding antibody chains include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

Each heavy chain of an antibody is comprised of a heavy chain variable region (V_{H}) and a heavy chain constant region. The heavy chain constant region of most IgG isotypes (subclasses) is comprised of three domains, C_{H1}, C_{H2} and C_{H3}, some IgG isotypes, like IgM or IgE comprise a fourth constant region domain, C_{H4} Each light chain is comprised of a light chain variable region (V_{L}) and a light chain constant region. The light chain constant region is comprised of one domain, C_{L}. The variable regions of the heavy and light chains contain a binding domain that interacts with an antigen. The constant regions of the antibodies may mediate the binding of the immunoglobulin to host tissues or factors, including various cells of the immune system and the first component (Clq) of the classical complement system.

The V_{H} and V_{L} regions of an antibody can be further subdivided into regions of hypervariability, also termed complementarity determining regions (CDRs), which are interspersed with the more conserved framework regions (FRs). Each V_{H} and V_{L} is composed of three CDRs and four FRs, arranged from amino-terminus to carboxyl-terminus in the following order: FR1, CDR1, FR2, CDR2, FR3, CDR3, FR4. The locations of CDR and FR regions and a numbering system have been defined by, e.g., Kabat et al., Sequences of Proteins of Immunological Interest, U.S. Department of Health and Human Services, U.S. Government Printing Office (1987 and 1991).

An “antibody-based binding protein”, as used herein, may represent any protein that contains at least one antibody-derived V_{H}, V_{L}, or CH immunoglobulin domain in the context of other non-immunoglobulin, or non-antibody derived components. Such antibody-based proteins include, but are not limited to (i) Fe-fusion proteins of binding proteins, including receptors or receptor components with all or parts of the immunoglobulin CH domains, (ii) binding proteins, in which V_{H} and or V_{L} domains are coupled to alternative molecular scaffolds, or (iii) molecules, in which immunoglobulin V_{H}, and/or V_{L}, and/or CH domains are combined and/or assembled in a fashion not normally found in naturally occurring antibodies or antibody fragments.

Antibody fragments (or “antigen-binding fragments”) refer to the antigen-binding portions of an intact antibody that retain capacity to bind the cognate antigen. Examples of such antibody fragments include (i) a Fab fragment, a monovalent fragment consisting of the V_{L}, V_{H}, C_{L} and C_{H1} domains; (ii) a F(ab)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an intact antibody; (v) disulfide stabilized Fvs (dsFvs) which have an interchain disulfide bond engineered between structurally conserved framework regions; (vi) a single domain antibody (dAb) which consists of a V_{H} domain (see, e.g., Ward et al., Nature 341:544-546, 1989); and (vii) an isolated complementarity determining region (CDR).

“Binding affinity” is generally expressed in terms of equilibrium association or dissociation constants (K_{A} or K_{D}, respectively), which are in turn reciprocal ratios of dissociation and association rate constants (k_{off} and kₒₙ, respectively). Thus, equivalent affinities may correspond to different rate constants, so long as the ratio of the rate constants remains the same. The binding affinity of an antibody is usually be expressed as the K_{D} of a monovalent fragment (e.g. a F_{ab} fragment) of the antibody, with K_{D} values in the single-digit nanomolar range or below (subnanomolar or picomolar) being considered as very high and of therapeutic and diagnostic relevance.

As used herein, the term “binding specificity” refers to the selective affinity of one molecule for another such as the binding of antibodies to antigens (or an epitope or antigenic determinant thereof), receptors to ligands, and enzymes to substrates. Thus, all monoclonal antibodies that bind to a particular antigenic determinant of an entity (e.g., a specific epitope of ROR1 or ROR2) are deemed to have the same binding specificity for that entity.

The term “conservatively modified variant” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences, “conservatively modified variants” refer to a variant which has conservative amino acid substitutions, amino acid residues replaced with other amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The term “contacting” has its normal meaning and refers to combining two or more agents (e.g., polypeptides or phage), combining agents and cells, or combining two populations of different cells. Contacting can occur in vitro, e.g., mixing an antibody and a cell or mixing a population of antibodies with a population of cells in a test tube or growth medium. Contacting can also occur in a cell or in situ, e.g., contacting two polypeptides in a cell by co-expression in the cell of recombinant polynucleotides encoding the two polypeptides, or in a cell lysate. Contacting can also occur in vivo inside a subject, e.g., by administering an agent to a subject for delivery the agent to a target cell.

A “humanized antibody” is an antibody or antibody fragment, antigen-binding fragment, or antibody-based binding protein comprising antibody V_{H} or V_{L} domains with a homology to human V_{H} or V_{L} antibody framework sequences having a T20 score of greater than 80, as defined by defined by Gao et al. (2013) BMC Biotechnol. 13, pp. 55.

The terms “identical” or percent “identity,” in the context of two or more nucleic acids or polypeptide sequences, refer to two or more sequences or subsequences that are the same. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

Methods of alignment of sequences for comparison are well known in the art. Optimal alignment of sequences for comparison can be conducted, e.g., by the local homology algorithm of Smith and Waterman, Adv. Appl. Math. 2:482c, 1970; by the homology alignment algorithm of Needleman and Wunsch, J. Mol. Biol. 48:443, 1970; by the search for similarity method of Pearson and Lipman, Proc. Nat'l. Acad. Sci. USA 85:2444, 1988; by computerized implementations of these algorithms (GAP, BESTFIT, FASTA, and TFASTA in the Wisconsin Genetics Software Package, Genetics Computer Group, Madison, Wis.); or by manual alignment and visual inspection (see, e.g., Brent et al., Current Protocols in Molecular Biology, John Wiley & Sons, Inc. (ringbou ed., 2003)). Two examples of algorithms that are suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively.

Programmed death-ligand 1 (PD-L1), also known as cluster of differentiation 274 (CD274) or B7 homolog 1 (B7-H1), is a protein that in humans is encoded by the CD274 gene. PD-L1 is a 40 kDa type 1 transmembrane protein, and is expressed in many types of human cancers, including in esophageal, gastrointestinal, pancreatic, breast, lung and kidney cancers. The binding of PD-L1 to the inhibitory checkpoint molecule PD-1 transmits an inhibitory signal based on interaction with phosphatases (SHP-1 or SHP-2) via Immunoreceptor Tyrosine-Based Switch Motif (ITSM) motif. This reduces the proliferation of antigen-specific T-cells in lymph nodes, while simultaneously reducing apoptosis in regulatory T cells (anti-inflammatory, suppressive T cells)—further mediated by a lower regulation of the gene Bcl-2.

The term “subject” refers to human and non-human animals (especially non-human mammals). The term “subject” is used herein, for example, in connection with therapeutic and diagnostic methods, to refer to human or animal subjects. Animal subjects include, but are not limited to, animal models, such as, mammalian models of solid tumors such as neuroblastoma, sarcoma, renal cell carcinoma, breast cancer, lung cancer, colon cancer, head and neck cancer, melanoma, and other cancers. Other specific examples of non-human subjects include, e.g., cows, horses, sheep, pigs, cats, dogs, mice, rats, rabbits, guinea pigs, monkeys.

The terms “treat,” “treating,” “treatment,” and “therapeutically effective” used herein do not necessarily imply 100% or complete treatment. Rather, there are varying degrees of treatment recognized by one of ordinary skill in the art as having a potential benefit or therapeutic effect. In this respect, the inventive method can provide any amount of any level of treatment. Furthermore, the treatment provided by the inventive method can include the treatment of one or more conditions or symptoms of the disease being treated.

A “vector” is a replicon, such as plasmid, phage or cosmid, to which another polynucleotide segment may be attached so as to bring about the replication of the attached segment. Vectors capable of directing the expression of genes encoding for one or more polypeptides are referred to as “expression vectors”.

### III. Tumor Targeting, Immune Checkpoint-Blocking Bispecific Molecules

The invention provides novel bispecific molecules that contain a PD-L1 targeting antibody, or an antibody-based binding protein or antibody fragment derived therefrom, and a peptide (or polypeptide) agent that can specifically bind to a cell surface antigen or molecular marker of a solid tumor. In the bispecific molecules of the invention, the peptide agent can be covalently or non-covalently conjugated to the antibody or antibody fragment. Preferably, the peptide agent is covalently linked to the antibody. In various embodiments, the antibody can be conjugated to the peptide agent using any type of suitable conjugation. For example, recombinant engineering and incorporated selenocysteine (e.g., as described in U.S. Pat. No. 8,916,159) can be used to conjugate the peptide agent. Other methods of conjugation can include covalent coupling to native or engineered lysine side-chain amines or cysteine side-chain thiols. See, e.g., Wu et al., Nat. Biotechnol, 23: 1 137-1 146 (2005).

In general, conjugation of the peptide agent to the antibody or antigen-binding fragment should not substantially affect the PD-L1 targeting function of the antibody. In some embodiments, the peptide agent is conjugated (e.g., covalently linked) to the antibody at a position that is outside the CDRs or the variable region of the antibody chains. In some embodiments, the peptide agent is linked to the constant region of the light chain of the antibody or antibody fragment. In some embodiments, the peptide agent is linked to the constant region of the heavy chain of the antibody or antibody fragment. In some of these embodiments, the peptide agent is linked to the Fc region of the antibody or antibody fragment. In some embodiments of the invention, the employed PD-L1 antibody does not contain a constant region, e.g., a single chain antibody or single domain antibody. In these embodiments, the peptide agent can be conjugated to the antibody at a position that will have the least impact on antigen recognition activity of the antibody, e.g., in the framework region of the variable domain of the antibody. In still some other embodiments, the employed antibody can tolerate insertion of a conjugated agent at the N-terminus or C-terminus of the antibody without substantially affecting its ability to bind PD-L1. In some of these embodiments, the peptide agent can be linked to the N-terminus of the antibody, as exemplified herein with PD-L1 antibody Atezolizumab. In some of these embodiments, the peptide agent is conjugated to the antibody at the N-terminus of an antibody chain via a suitable linker. In some of these embodiments, the conjugation is at a heavy chain N-terminus of the PD-L1 antibody through an engineered N-terminal attachment site, e.g., an engineered serine residue as exemplified herein.

Any suitable linking moieties can be employed in the conjugation of the peptide agent to the antibody. In various embodiments, the linker moiety can be an oligopeptide linker (including cleavable and non-cleavable oligopeptide linkers), chemical moieties that link via click chemistry, a hydrazine linker, a thiourea linker, a self-immolative linker, a succinimidyl trans-4-(maleimidylmethyl)cyclohexane-1-carboxylate (SMCC) linker, a maleimide linker, a disulfide linker, a thioether linker, and/or a maleimide linker. The skilled artisan would understand that many other linkers may also be suitable for the invention. In various embodiments, the linkers may be non-cleavable or may be cleaved by changes in pH, redox potential or specific intracellular enzymes. Cleavable oligopeptide linkers include protease- or matrix metalloprotease-cleavable linkers. It is understood that the linker may comprise combinations of the above. For example, the linker may be a valine-citruline PAB linker. In some embodiments, conjugation of the peptide agent to the PD-L1 antibody is achieved by linking moieties that react via click chemistry. As exemplified herein, a NHS-BCN linker compound can be used to label the PD-L1 targeting antibody for conjugation of the peptide agent. With this linking compound, the NHS ester can react with the primary amine (—NH₂) of an amino acid residue in the antibody (e.g., a Lys residue in the Fc region). This is followed by reacting the BCN group with azide-tagged peptide agent by click chemistry. In some other embodiments, a short peptide or oligopeptide linker can be used to link the peptide agent to the heavy chain or light chain of the PD-L1 targeting antibody. As exemplified herein, a serine containing peptide linker such as a G₄S (GGGGS; SEQ ID NO:2) # linker can be attached to the N-terminus of a heavy chain of PD-L1 antibody Atezolizumab for conjugation of a MC1R-targeting peptide agent (e.g., NDP-MSH). In various embodiments, the peptide linker can contain 1, 2, 3, 4, 5, 6, 7, 8, 9, 10 or more tandem repeats of G₄S. In these embodiments, the conjugation can be achieved by, e.g., recombinant techniques.

Any PD-L1 targeting antibody can be employed in the practice of the invention. Preferably, the employed antibody or antigen-binding fragment is monoclonal. In some embodiments, the employed PD-L1 antibody is reactive with human PD-L1. In some embodiment, the employed PD-L1 antibody is a human antibody, a humanized antibody or a chimeric antibody. These include several monoclonal PD-L1 antibodies that have been approved for various human therapies. For example, Atezolizumab (Tecentriq) is a fully humanized IgG1 antibody developed by Roche Genentech. It was approved by the FDA for urothelial carcinoma and non-small cell lung cancer. Avelumab (Bavencio) is a fully human IgG1 antibody developed by Merck Serono and Pfizer, and is approved by FDA approved for the treatment of metastatic merkel-cell carcinoma. Durvalumab (Imfinzi) is a fully human IgG1 antibody developed by AstraZeneca, and is approved by the FDA for the treatment of urothelial carcinoma and unresectable non-small cell lung cancer after chemoradiation. As exemplified herein with Avelumab and Atezolizumab, any of these human or humanized antibodies, or antigen-binding fragments (antibody fragments) derived therefrom, can be used in the practice of the invention.

Many other PD-L1 antibodies are also known in the art. See, e.g., US Patent/Publication Nos. 7943742, 8383796, 8217149, 20090055944, 20120003056, 20130034559, 20130045200, 20130045201, 20130045202, 20170158767, 20180196055, and 20190106494, and also WO2007005874, WO2011066389, WO2010077634, WO2015112805, EP1907424, and EP1899379. Any of these antibodies, including antibody fragments thereof, may also be used in constructing the bispecific molecules of the invention.

In the practice of the invention, suitable PD-L1 antibodies or antigen-binding fragments include intact antibodies (e.g., IgG1 antibodies exemplified herein), antibody fragments or antigen-binding fragments (e.g., Fab fragments), and antibody-based binding proteins that contain the antigen-binding portions of an intact antibody that retain capacity to bind the cognate antigen, PD-L1. Examples of such antibody fragments include (i) a Fab fragment, a monovalent fragment consisting of the V_{L}, V_{H}, C_{L} and C_{H1} domains; (ii) a F(ab′)2 fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an intact antibody; (v) disulfide stabilized Fvs (dsFvs) which have an interchain disulfide bond engineered between structurally conserved framework regions; (vi) a single domain antibody (dAb) which consists of a V_{H} or V_{L} domain (see, e.g., Ward et al., Nature 341:544-546, 1989); and (vii) an isolated complementarity determining region (CDR) as a linear or cyclic peptide. Other examples of antibody-based binding proteins include polypeptides in which the binding domains of the antibodies are combined with other polypeptides or polypeptide domains, e.g. alternative molecular scaffolds, Fc-regions, other functional or binding domains of other polypeptides or antibodies resulting in molecules with addition binding properties, e.g. bi- or multispecific proteins or antibodies. Such polypeptides can create an arrangement of binding or functional domains normally not found in naturally occurring antibodies or antibody fragments.

In some embodiments of the invention, the employed PD-L1 targeting antibodies are antibody fragments (or “antigen-binding fragments”), like single chain antibodies. The term “single chain antibody” refers to a polypeptide comprising a V_{H} domain and a V_{L} domain in polypeptide linkage, generally linked via a spacer peptide, and which may comprise additional domains or amino acid sequences at the amino- and/or carboxyl-termini. For example, a single-chain antibody may comprise a tether segment for linking to the encoding polynucleotide. As an example, a single chain variable region fragment (scFv) is a single-chain antibody. Compared to the V_{L} and V_{H} domains of the Fv fragment which are coded for by separate genes, a scFv has the two domains joined (e.g., via recombinant methods) by a synthetic linker. This enables them to be made as a single protein chain in which the V_{L} and V_{H} regions pair to form monovalent molecules.

In some embodiments, the employed PD-L1 targeting antibodies for the present invention are single domain antigen-binding units, which have a camelid scaffold. Animals in the camelid family include camels, llamas, and alpacas. Camelids produce functional antibodies devoid of light chains. The heavy chain variable (V_{H}) domain folds autonomously and functions independently as an antigen-binding unit. Its binding surface involves only three CDRs as compared to the six CDRs in classical antigen-binding molecules (Fabs) or single chain variable fragments (scFvs). Camelid antibodies are capable of attaining binding affinities comparable to those of conventional antibodies.

In some preferred embodiments, the employed PD-L1 antibodies for practicing the invention are human antibodies or humanized antibodies with higher homology at amino acid level of the humanized antibody V_{H} or V_{L} domains to human antibody V_{H} or V_{L} domains than rodent V_{H} or V_{L} domains, preferably with a T20 score of greater than 80 as defined by Gao et al. (2013) BMC Biotechnol. 13, pp. 55. In some of these embodiments, the employed PD-L1 antibody is Avelumab, Durvalumab or Atezolizumab, an antigen-binding fragment thereof, or other variants with the same or substantially identical binding properties (e.g., affinity and/or specificity). In some embodiments, variants of the known PD-L1 antibodies include variants that contain one or more conservative amino acid substitutions.

In some embodiments, the employed antibodies, antibody fragments, or antibody-based binding proteins can have heavy chain CDR1, CDR2 and CDR3 sequences and light chain CDR1, CDR2 and CDR3 sequences that are substantially identical to that of a known PD-L1 targeting antibody described herein. In some of these embodiments, the employed antibody can have heavy chain CDR1-CDR3 and light chain CDR1-CDR3 sequences that are identical to one of the known PD-L1 targeting antibodies except for conservative substitutions of one or more amino acid residues. In some embodiments, the employed antibody can have a light chain variable domain sequence and/or a heavy chain variable sequence that are substantially identical to the light chain variable domain sequence and heavy chain variable sequence, respectively, of a known PD-L1 targeting antibody. In some embodiments, the percentage identity can be at least 91%, at least 92%, at least 93%, at least 94%, at least 95%, at least 96%, at least 97%, at least 98%, or at least 99%, or even 100%. In some embodiments, the employed antibodies, antibody fragments, or antibody-based binding proteins can be conservatively modified variants, i.e., variants that contain at least one conservatively modified residue relative to the sequence of the reference antibody (e.g., Atezolizumab, Avelumab or Durvalumab). For example, the variants can have one or more conservatively modified residues in the constant region of the reference antibody, in the framework region of the heavy chain or light variable domain, or even in one or more of the heavy chain or light chain CDRs. Preferably, relative to the reference antibody, the conservative modified variants should have substantially the same binding specificity and/or the same or better binding affinity for the cognate target molecule.

The various PD-L1 targeting antibodies, antibody-binding proteins or antibody fragments thereof described herein may be purchased from commercial suppliers or can be produced by enzymatic or chemical modification of the intact antibodies, or synthesized de novo using recombinant DNA methodologies, or identified using phage display libraries. As exemplified herein, genes encoding the variable regions sequences of PD-L1 antibodies can be obtained from vendors such as Integrated DNA Technologies, Inc. (IDT) and amplified via standard PCR techniques. Other suitable methods for generating the antibodies or antigen-binding fragments are all well known in the art. For example, single chain antibodies can be identified using phage display libraries or ribosome display libraries, gene shuffled libraries (see, e.g., McCafferty et al., Nature 348:552-554, 1990; and U.S. Pat. No. 4,946,778). In particular, scFv antibodies can be obtained using methods described in, e.g., Bird et al., Science 242:423-426, 1988; and Huston et al., Proc. Natl. Acad. Sci. USA 85:5879-5883, 1988. Fv antibody fragments can be generated as described in Skerra and Plückthun, Science 240:1038-41, 1988. Disulfide-stabilized Fv fragments (dsFvs) can be made using methods described in, e.g., Reiter et al., Int. J. Cancer 67:113-23, 1996. Similarly, single domain antibodies (dAbs) can be produced by a variety of methods described in, e.g., Ward et al., Nature 341:544-546, 1989; and Cai and Garen, Proc. Natl. Acad. Sci. USA 93:6280-85, 1996. Camelid single domain antibodies can be produced using methods well known in the art, e.g., Dumoulin et al., Nat. Struct. Biol. 11:500-515, 2002; Ghahroudi et al., FEBS Letters 414:521-526, 1997; and Bond et al., J. Mol. Biol. 332:643-55, 2003. Other types of antigen-binding fragments (e.g., Fab, F(ab′)₂ or Fd fragments) can also be readily produced with routinely practiced immunology methods.

The various PD-L1 targeting antibodies or antibody fragments for use in the invention can also be produced by any suitable technique, for example, using any suitable eukaryotic or non-eukaryotic expression system. See, e.g., Harlow & Lane, Using Antibodies, A Laboratory Manual, Cold Spring Harbor Laboratory Press, Cold Spring Harbor, N.Y., 1998. In some embodiments, the antibodies or antigen-binding fragments can be produced via a mammalian expression system. Some specific techniques for generating the antibodies antibody-based binding proteins or antibody fragments thereof of the invention are exemplified herein, e.g., the FreeStyle 293-F cell expression system.

In various embodiments, the peptide or polypeptide agents for constructing the bispecific molecules of the invention encompass any naturally existing or synthetic polypeptides or peptides that are capable of specifically binding to a cell surface antigen or molecule marker of solid tumors. These include, e.g., ligands and analogs of some tyrosine kinase receptors (e.g., EGFR, FGFR, NGFR and ephrin receptors) that are up-regulated in many types of cancer. Other tumor surface antigens or molecular markers that can be targeted with the bispecific molecules of the invention include, e.g., melanocortin 1 receptor (MC1R), a G protein-coupled receptor that is located on the plasma membrane of melanocytes. Many ligands that specifically recognize these solid tumor surface antigens are well known in the art. For example, MC1R is bound by a class of pituitary peptide hormones known as the melanocortins, which include adrenocorticotropic hormone (ACTH) and the different forms of melanocyte-stimulating hormone (MSH). Other examples of peptide agents for targeting tumor markers include, e.g., EGF, amphiregulin, heparin-binding EGF-like growth factor, epiregulin, transforming growth factor-α and β-cellulin. See, e.g., Kolonin et al., Cancer Res. 2006; 66:34-40.

Some embodiments of the invention are directed to bispecific molecules that target melanoma. In some of these embodiments, the employed peptide agent is a known ligand of a cell surface marker of melanoma. For example, the peptide agent can be α-MSH, a hormone ligand of the MC1R receptor expressed on melanoma cells. In some other embodiments, the employed peptide agent can be a synthetic analog (e.g., NDP-MSH) of the natural ligand. For targeting MC1R in melanoma, suitable MSH analogues can also include any of the other known analogues of the ligand α-MSH or α-MSH. See, e.g., Fung et al., Curr Opin Chem Biol. 2005, 9:352-358; Grieco et al., PLoS One. 2013, 8(4):e61614; and Zhou et al., J. Med. Chem. 2017, 60, 9320-9329.

In addition to the various known peptide or polypeptide agents, their variants, analogs or derivatives that share the same binding functions can also be used in the practice of the invention. These include, e.g., variants peptides or polypeptides with sequences that are substantially identical (e.g., at least 75%, 80%, 85%, 90%, 95%, or 99% identical) to that of the reference peptide or polypeptide agent (e.g., α-MSH). In some of these embodiments, the employed variant peptide or polypeptide agent can be one that contains at least one (e.g., at least 1, 2, 3, 4, 5, 6, 7, 8, 9, 10, 15, 20 or more) conservatively modified residue relative to the reference peptide or polypeptide, i.e., a conservatively modified variant.

### IV. Polynucleotides Chains or Domains of the Bispecific Molecules

The invention provides substantially purified polynucleotides (DNA or RNA) that are identical or complementary to sequences encoding polypeptides comprising chains, segments or domains of the bispecific molecules of the invention. Also provided in the invention are expression vectors and host cells for producing chains, segments or domains of some bispecific functional antibodies described herein that are generated via recombinant means. Specific examples of vectors and host cells are exemplified herein. Various other expression vectors can also be employed to express the polynucleotides encoding the functional antibody chains or binding fragments. Both viral-based and nonviral expression vectors can be used to produce the antibodies in a mammalian host cell. Nonviral vectors and systems include plasmids, episomal vectors, typically with an expression cassette for expressing a protein or RNA, and human artificial chromosomes (see, e.g., Harrington et al., Nat. Genet. 15:345, 1997). For example, nonviral vectors useful for expression of the antibody polynucleotides and polypeptides in mammalian (e.g., human) cells include pCEP4, pREP4, pThioHis A, B & C, pcDNA3.1/His, pEBVHis A, B & C (Invitrogen, San Diego, Calif.), MPSV vectors, and numerous other vectors known in the art for expressing other proteins. Other useful nonviral vectors include vectors that comprise expression cassettes that can be mobilized with Sleeping Beauty, PiggyBack and other transposon systems. Useful viral vectors include vectors based on lentiviruses or other retroviruses, adenoviruses, adeno-associated viruses, herpes viruses, vectors based on SV40, papilloma virus, HBP Epstein Barr virus, vaccinia virus vectors and Semliki Forest virus (SFV). See, Brent et al., supra; Smith, Annu. Rev. Microbiol. 49:807, 1995; and Rosenfeld et al., Cell 68:143, 1992.

The host cells for harboring and expressing the functional bispecific antibody chains can be either prokaryotic or eukaryotic. In some preferred embodiments, mammalian host cells are used to express and to produce the antibody polypeptides of the present invention. For example, they can be either a hybridoma cell line expressing endogenous immunoglobulin genes or a mammalian cell line harboring an exogenous expression vector. These include any normal mortal or normal or abnormal immortal animal or human cell. In addition to the cell lines exemplified herein, a number of other suitable host cell lines capable of secreting intact immunoglobulins are also known in the art. These include, e.g., the CHO cell lines, various HEK 293 cell lines, various Cos cell lines, HeLa cells, myeloma cell lines, transformed B-cells and hybridomas. The use of mammalian tissue cell culture to express polypeptides is discussed generally in, e.g., Winnacker, From Genes to Clones, VCH Publishers, N.Y., N.Y., 1987. Expression vectors for mammalian host cells can include expression control sequences, such as an origin of replication, a promoter, and an enhancer, and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, EF1α and human UbC promoters exemplified herein, the metallothionein promoter, the constitutive adenovirus major late promoter, the dexamethasone-inducible MMTV promoter, the SV40 promoter, the MRP pol III promoter, the constitutive MPSV promoter, the tetracycline-inducible CMV promoter (such as the human immediate-early CMV promoter), the constitutive CMV promoter, and promoter-enhancer combinations known in the art.

Methods for introducing expression vectors containing the polynucleotide sequences of interest vary depending on the type of cellular host. For example, calcium chloride transformation is commonly utilized for prokaryotic cells, whereas calcium phosphate treatment or electroporation may be used for other cellular hosts (see generally Sambrook et al., supra). Other methods include, e.g., electroporation, calcium phosphate treatment, liposome-mediated transformation, injection and microinjection, ballistic methods, virosomes, immunoliposomes, polycation:nucleic acid conjugates, naked DNA, artificial virions, fusion to the herpes virus structural protein VP22 (Elliot and O'Hare, Cell 88:223, 1997), agent-enhanced uptake of DNA, and ex vivo transduction. For long-term, high-yield production of recombinant proteins, stable expression will often be desired. For example, cell lines which stably express the antibody chains or binding fragments can be prepared using expression vectors of the invention which contain viral origins of replication or endogenous expression elements and a selectable marker gene. Following introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate for the cell type.

### V. Therapeutic Applications and Related Pharmaceutical Compositions

The tumor targeting bispecific molecules of the invention can be used in various therapeutic or prophylactic applications. Depending on the target of the peptide agent in the bispecific molecules, various types of tumors can be treated or prevented with the bispecific molecules of the invention. In various embodiments, tumors that can be treated include, e.g., melanoma, breast cancer, lung cancer, colon cancer, neuroblastoma, sarcoma, renal cell carcinoma, head and neck cancer. Typically, therapeutic methods of the invention entail administration of a bispecific molecule described herein to a subject that has, is suspected to have, or is at risk of developing a tumor that expresses a cellular marker that can be targeted by the peptide agent in the employed bispecific molecule. In some embodiments, a subject afflicted with or at risk of developing melanoma can be treated with bispecific molecules of the invention. For example, the MC1R targeting bispecific molecule exemplified herein can be readily employed for treating, slowing the progress or preventing the development of melanoma in a subject.

In some embodiments, the bispecific molecules of the invention can be used with other therapeutic agent in combination therapies for tumors. For example, the bispecific molecules can be used together with other immune checkpoint inhibitors, cytotoxic agents, cytostatic agents, antiangiogenic agents or therapeutic radioisotopes. In some of these embodiments, the method can include co-administration of a cytotoxic, cystostatic, or antiangiogenic or immune-stimulatory agent (e.g. immune-checkpoint inhibitor antibodies, for instance, but not limited to, those binding to PD1, PDL1, CTLA4, OX40, TIM3, GITR, LAG3 and the like) suitable for treating the cancer. Thus, in various embodiments for treating melanoma, the melanoma targeting bispecific molecules described herein can be used in combination with, e.g., PD-1 inhibitors such as Pembrolizumab (Keytruda) and nivolumab (Opdivo), CTLA-4 inhibitor such as Ipilimumab (Yervoy), or cytokines such as interferon α and IL-2α. Similarly, the bispecific molecules described herein for targeting other types of tumors can be used in combination with known therapies for treating the respective tumors, e.g., esophageal, gastrointestinal, pancreatic, breast, lung and kidney cancers.

For use in the therapeutic methods described herein, the invention also provides pharmaceutical compositions that contain a bispecific molecule of the invention and a pharmaceutically acceptable carrier. Pharmaceutical compositions can be prepared from any of the bispecific molecules described herein, e.g., a melanoma targeting bispecific molecule containing the PD-L1 antibody Avelumab or Atezolizumab as exemplified herein. The pharmaceutically acceptable carrier can be any suitable pharmaceutically acceptable carrier. It can be one or more compatible solid or liquid fillers, diluents, other excipients, or encapsulating substances which are suitable for administration into a human or veterinary patient (e.g., a physiologically acceptable carrier or a pharmacologically acceptable carrier). The term “carrier” denotes an organic or inorganic ingredient, natural or synthetic, with which the active ingredient is combined to facilitate the use of the active ingredient, e.g., the administration of the active ingredient to a subject. The pharmaceutically acceptable carrier can be co-mingled with one or more of the active components, e.g., a hybrid molecule, and with each other, when more than one pharmaceutically acceptable carrier is present in the composition, in a manner so as not to substantially impair the desired pharmaceutical efficacy. Pharmaceutically acceptable materials typically are capable of administration to a subject, e.g., a patient, without the production of significant undesirable physiological effects such as nausea, dizziness, rash, or gastric upset. It is, for example, desirable for a composition comprising a pharmaceutically acceptable carrier not to be immunogenic when administered to a human patient for therapeutic purposes.

Pharmaceutical compositions of the invention can additionally contain suitable buffering agents, including, for example, acetic acid in a salt, citric acid in a salt, boric acid in a salt, and phosphoric acid in a salt. The compositions can also optionally contain suitable preservatives, such as benzalkonium chloride, chlorobutanol, parabens, and thimerosal. Pharmaceutical compositions of the invention can be presented in unit dosage form and can be prepared by any suitable method, many of which are well known in the art of pharmacy. Such methods include the step of bringing the antibody of the invention into association with a carrier that constitutes one or more accessory ingredients. In general, the composition is prepared by uniformly and intimately bringing the active agent into association with a liquid carrier, a finely divided solid carrier, or both, and then, if necessary, shaping the product.

A composition suitable for parenteral administration conveniently comprises a sterile aqueous preparation of the inventive composition, which preferably is isotonic with the blood of the recipient. This aqueous preparation can be formulated according to known methods using suitable dispersing or wetting agents and suspending agents. The sterile injectable preparation also can be a sterile injectable solution or suspension in a non-toxic parenterally-acceptable diluent or solvent, for example, as a solution in 1,3-butane diol. Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil can be employed, such as synthetic mono- or di-glycerides. In addition, fatty acids such as oleic acid can be used in the preparation of injectables. Carrier formulations suitable for oral, subcutaneous, intravenous, intramuscular, etc. administrations can be found, e.g., in Remington: The Science and Practice of Pharmacy, Mack Publishing Co., 20^{th} ed., 2000.

Preparation of pharmaceutical compositions of the invention and their various routes of administration can be carried out in accordance with methods well known in the art. See, e.g., Remington, supra; and Sustained and Controlled Release Drug Delivery Systems, J. R. Robinson, ed., Marcel Dekker, Inc., New York, 1978. The delivery systems useful in the context of the invention include time-released, delayed release, and sustained release delivery systems such that the delivery of the inventive composition occurs prior to, and with sufficient time to cause, sensitization of the site to be treated. The inventive composition can be used in conjunction with other therapeutic agents or therapies. Such systems can avoid repeated administrations of the inventive composition, thereby increasing convenience to the subject and the physician, and may be particularly suitable for certain compositions of the invention.

Many types of release delivery systems are available and known to those of ordinary skill in the art. Suitable release delivery systems include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are lipids including sterols such as cholesterol, cholesterol esters, and fatty acids or neutral fats such as mono-di- and triglycerides; hydrogel release systems; sylastic systems; peptide based systems; wax coatings; compressed tablets using conventional binders and excipients; partially fused implants; and the like. Specific examples include, but are not limited to: (a) erosional systems in which the active composition is contained in a form within a matrix such as those described in U.S. Pat. Nos. 4,452,775, 4,667,014, 4,748,034, and 5,239,660 and (b) diffusional systems in which an active component permeates at a controlled rate from a polymer such as described in U.S. Pat. Nos. 3,832,253 and 3,854,480. In addition, pump-based hardware delivery systems can be used, some of which are adapted for implantation.

### Examples

The following examples are provided to further illustrate the invention but not to limit its scope. Other variants of the invention will be readily apparent to one of ordinary skill in the art and are encompassed by the appended claims.

### Example 1. Construction of NDP-MSH-αPD-L1 Conjugate

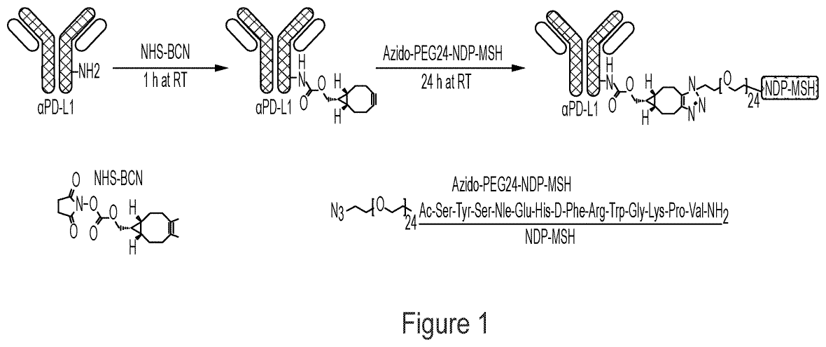

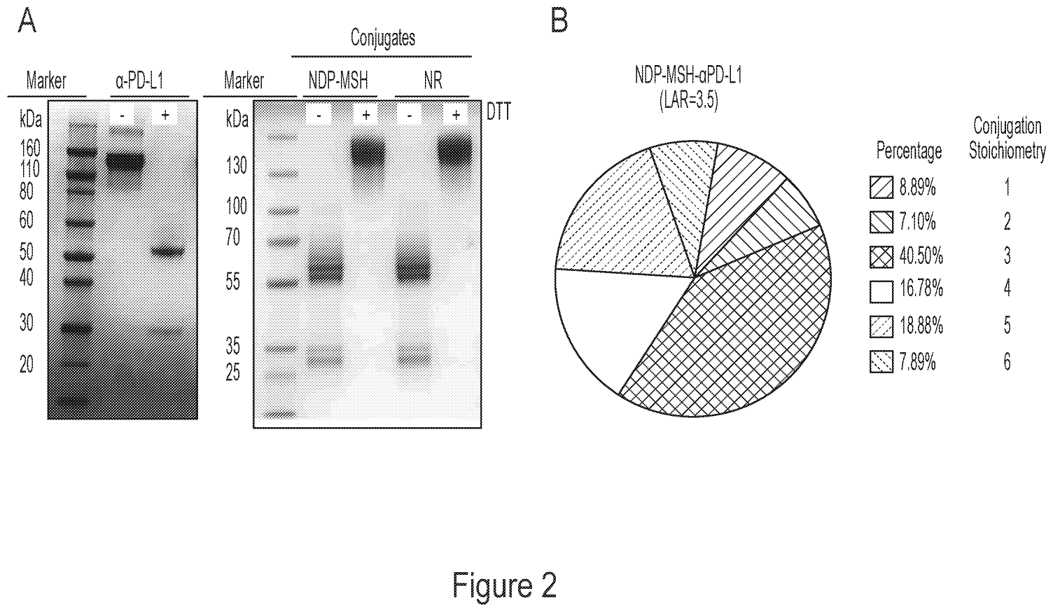

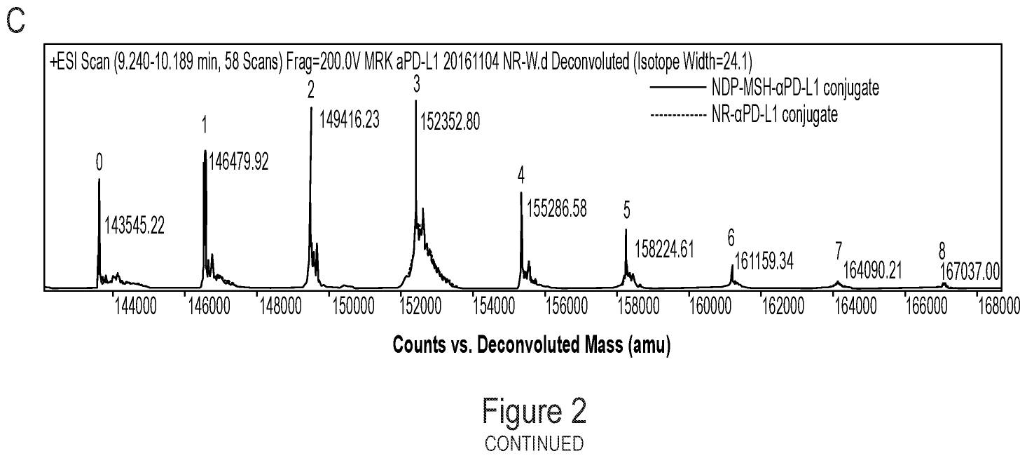

As a marker of melanoma risk, MC1R is expressed at significantly higher levels in more than 80% of human melanomas. Over the years, radiolabeled α-MSH (a natural ligand of MC1R) and its analogues have been used for melanoma imaging and treatment. Therefore, we initially selected α-MSH as a targeting agent, and α-MSH analogues were chemically conjugated to the anti-PD-L1 monoclonal antibody, Avelumab. To generate a bispecific-antibody, a potent MSH analog [Nle4, D-Phe7]-MSH (NDP-MSH) with a PEG linker (Azido-PEG24-SYS-Nle-EHfRWGKPV-CONH2, Nle=Norleucine, f=D-form Phe) was synthesized (FIG. 1). This biologically stable synthetic MSH analog was approved in Europe in 2015 to prevent UV skin damage in people with erythropoietic protoporphyria (EPP) and has a higher binding affinity to MC1R than α-MSH (0.67±0.09 nM vs 2.58±0.33 nM), which helps overcome in vivo competition by endogenous ligand. This peptide showed high shelf stability and good biological stability in vivo. A peptide with a similar but non-binding sequence was also synthesized and used as a control (NR, Azido-PEG24-SEGYHKSfRP-Nle-WV-CONH2). The human IgG1 αPD-L1 antibody, Avelumab, is human and mouse cross reactive (K_{d}=0.3 nM and 1 nM, respectively), and therefore was chosen as the antibody backbone. Heavy and light chain genes of Avelumab were cloned into pFuse vector and co-expressed by transient transfection in FreeStyle 293F cells in a yield of 30 mg/L. SDS/PAGE analysis revealed >90% purity (FIG. 2A). After reduction by dithiothreitol (DTT), the light chains migrated at 25 kDa and the heavy chain migrated at 50 kDa, matching the calculated molecular mass of heavy and light chains.

The anti-PD-L1/NDP-MSH (NDP-MSH-αPD-L1) bispecific antibody was generated by nonspecifically conjugating an NHS ester of NDP-MSH to lysine residues of the αPD-L1 antibody by a two-step ligation (FIG. 1). Briefly, NHS-BCN was conjugated to the primary amine of exposed lysines of αPD-L1 antibody (1 mg/ml) in phosphate-buffered saline (PBS) at pH 8.3 for 1 h at room temperature to form stable amide bonds. After removing unreacted NHS-BCN using a desalting column, the BCN-conjugated αPD-L1 antibody (0.8 mg/ml) was then reacted with azido-PEG24-NDP-MSH (or —NR) by a catalyst-free “click reaction” in a 1:20 molar ratio at pH 7.0 and 37° C. for 24 h. The product was purified by size-exclusion chromatography to remove excess non-conjugated NDP-MSH peptide. The antibody conjugates were analyzed by SDS/PAGE under reducing and non-reducing conditions. After reduction by DTT, the light chains migrated at 25-35 kDa and the heavy chains migrated at 50-65 kDa, in the form of multiple bands with ˜3 kDa increment between each band (FIG. 2A). The antibody is 90% conjugated with stoichiometries ranging from 1 to 8 MSH-peptide/antibody as determined by mass spectrometry analysis with expected molecular weights (FIG. 2C). The average MSH ligand to antibody ratio (LAR) is about 3.5 based on mass spectroscopy analysis (FIG. 2B). The anti-PD-L1/NR (NR-αPD-L1) was generated and analyzed by the same methods. The overall yields for the purified conjugated product range from 30-40% and the conjugate can be concentrated to 12 mg/ml without aggregation.

### Example 2. In Vitro Activities of NDP-MSH-αPD-L1 Conjugate

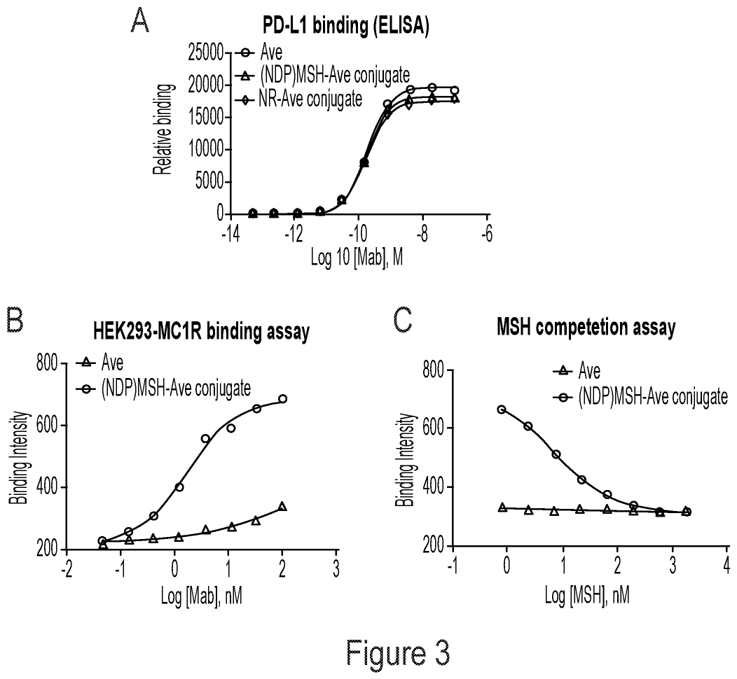

Next we characterized the binding of the conjugate to its respective receptors. NDP-MSH-αPD-L1 and NR-αPD-L1 show nearly the same binding affinity (EC₅₀=0.17±0.02 nM and 0.18±0.01 nM, respectively) to a human PD-L1 (extracellular domain)-Fc fusion protein by ELISA as that of αPD-L1 antibody alone (EC₅₀=0.19±0.01 nM) (FIG. 3A). This result indicates that an LAR=3.5 does not significantly affect binding of the conjugated antibody to PD-L1. The binding of NDP-MSH-αPD-L1 to human MC1R was analyzed by cell surface ELISA with a HEK293 cell line that overexpresses human MC1R as reported in Yang et al., Molecular and Cellular Endocrinology 454:69-76, 2017. NDP-MSH-αPD-L1 bound HEK293-MC1R cells in a dose-dependent manner (EC₅₀=1.72±0.31 nM) (FIG. 3B), and this specific binding was competed by free MSH peptide (FIG. 3C). The activities of the NDP-MSH-αPD-L1 conjugates were also examined using HEK 293 cells overexpressing MC1R and carrying a cAMP response element (CRE) luciferase (Luc) reporter. Cell surface MC1Rs were activated by NDP-MSH-αPD-L1 dose-dependently, and downstream signal transduction was induced with an EC₅₀=2.70±1.03 nM, similar to the value from the cell surface ELISA assay. This result indicates that NDP-MSH-αPD-L1 can activate MC1R with nanomolar potency, similar to that of Azido-PEG24-NDP-MSH (EC₅₀=0.94±0.11 nM), but less than that of the NDP-MSH peptide (EC₅₀=0.09±0.02 nM) in this cell-based reporter assay. Given the similar EC₅₀s of Azido-PEG24-NDP-MSH and the antibody conjugate, this reduced affinity to MC1R likely results from the linker at the N-terminus of NDP-MSH interfering to some degree with engagement of MC1R.

Avelumab is cross-reactive with human and mouse PD-L1, and therefore is suitable for both in vivo efficacy studies in syngeneic mouse models and ultimately human clinical studies. Likewise, NDP-MSH binds to both human and mouse MC1R. We further confirmed binding of the conjugate NDP-MSH-αPD-L1 to mouse B16-SIY cells (a melanoma cell line derived from B16) that highly express mouse PD-L1 and MC1R. Incubation of 500 nM αPD-L1 with B16-SIY cells resulted in a peak shift in flow cytometry analysis. Similar binding was observed with NDP-MSH-αPD-L1 and NR-αPD-L1. These results demonstrate that the bispecific conjugate can bind both MC1R and PD-L1 in vitro with good affinity, and suggests that the B16-SIY mouse melanoma model can be used to investigate its efficacy.

### Example 3. Serum Stability and Pharmacokinetic Analysis of NDP-MSH-αPD-L1

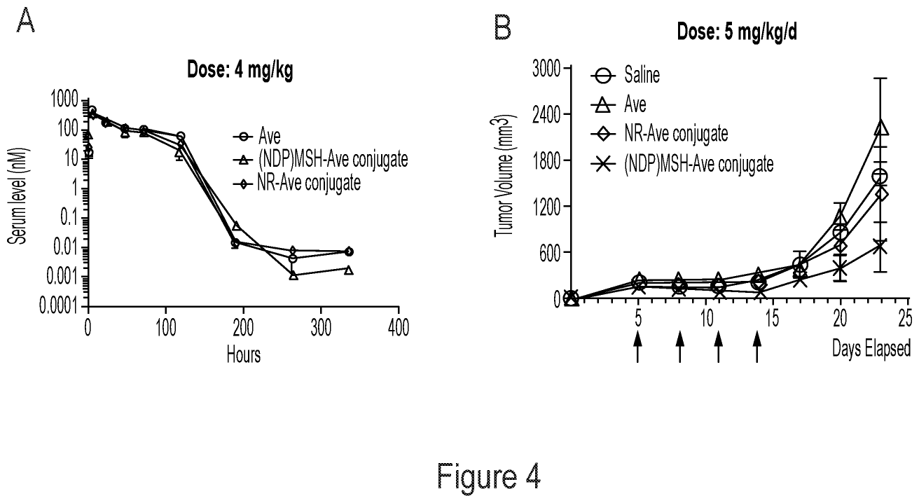

The stability of NDP-MSH-αPD-L1 was examined in freshly collected mouse serum. The concentration of the conjugated antibody was determined by ELISA using the PD-L1(ECD)-Fc fusion antigen. During 72 h of incubation, no significant degradation was observed, suggesting that peptide conjugation does not reduce the stability of the antibody in mouse serum. In addition, NDP-MSH-αPD-L1 has a melting temperature at 64° C. in a thermal stability assay, similar to that of αPD-L1. We next performed a pharmacokinetic (PK) analysis of NDP-MSH-αPD-L1 in mice, analyzing plasma samples using the same ELISA method described above in serum stability assay. NDP-MSH-αPD-L1, NR-αPD-L1, and αPD-L1 antibody show a similar PK profile after intraperitoneal injection, with terminal half-lives ranging from 16 to 19 h (FIG. 4A), which is typical for a human IgG in mice.

### Example 4. In Vivo Efficacy of NDP-MSH-αPD-L1 Conjugate

B16-F10 murine melanoma-bearing model was utilized for the studies of MC1R-targeted radiotherapies. B16-SIY cells were derived from B16-F10 expressing an engineered model antigen SIYRYYGL (SIY), which are more immunogenic than B16 cells and responsive to αPD-L1 treatment. Therefore we chose B16-SIY cells to develop a mouse MC1R⁺/PD-L1⁺ melanoma syngeneic model and used it to compare the in vivo efficacy of αPD-L1, NR-αPD-L1 and NDP-MSH-αPD-L1. Specifically, C57BL/6 mice were s.c. inoculated with 1.5×10⁶ B16-SIY tumor cells on day 0, and treatment was initiated on day 5 post injection when the tumor volume reached ˜100 mm³. Treatment consisted of four intraperitoneal injections every two days. Groups of mice were treated at doses of 1 mg/kg and 5 mg/kg for each construct (n=10/group). The control group was treated with saline only. As shown in FIG. 4B, treatment with NDP-MSH-αPD-L1 exhibited a significant antitumor effect. Mice treated with the 5 mg/kg dose of NDP-MSH-αPD-L1 exhibited a strong tumor growth inhibition (p<0.05 on days 23), In the 5 mg/kg NDP-MSH-αPD-L1 treatment group, tumor sizes in 80% of mice were under 500 mm³, and 20% of mice showed tumor regression during the treatment time. In mice treated with 1 mg/kg, tumor growth was slowed for the duration of the treatment. In contrast, treatment of mice with 5 mg/kg αPD-L1 antibody or NR-αPD-L1 showed no significant antitumor effect beyond that observed with saline only (p=0.174 and 0.345 on days 23, respectively).

To gain a better understanding of how this bispecific antibody reduced tumor load, we examined whether the number of mouse T cells in the tumor environment correlated with tumor growth inhibition by NDP-MSH-αPD-L1. Tumors were harvested on day 23, and cells were isolated from solid tumors by enzymatic digestion. The T-cell population within the tumor was analyzed by flow cytometry. After staining with a mouse CD3 surface marker, the results confirmed that a significantly higher percentage of CD3⁺ T cells accumulated in tumor tissue after 5 mg/kg NDP-MSH-αPD-L1 treatment (1.8±1.9%) as compared to groups treated with αPD-L1 (0.45±0.6%) antibody or NR-αPD-L1 (0.8±0.46%).

### Example 5. Generation and Characterization of N-Terminal Conjugated αPD-L1 Bispecific Molecules

In addition to αPD-L1 bispecific molecules with a MC1R-targeting agent conjugated at the constant region, we also generated additional MSH-αPD-L1 conjugates targeting melanoma by site-specific conjugation at the N-terminus via an engineered serine residue. Since there is only one engineered serine in a heavy chain of the antibody, the resulting conjugate is homogeneous and the ligand to antibody ratio (LAR) is 2. This is unlike some other conjugation schemes, e.g., conjugation via lysine residue, which could lead to heterogeneous conjugation products due to the fact that there are several exposed lysine residues on the antibody and that all of the residues can potentially react with the conjugation partner.

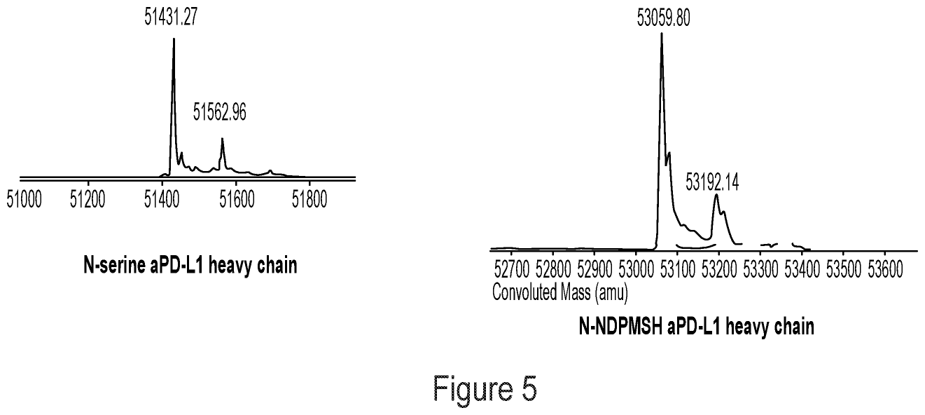

Specifically, we used PD-L1 antibody Atezolizumab which allows N-terminal conjugation without impact on PD-L1 binding. We first genetically engineered a serine residue followed by a 8× (G4S) linker at the N-terminal of heavy chain domain and recombinant expressed it in 293F cells. Then the 2-amino alcohol of the terminal serine residue was oxidized to an aldehyde by sodium periodate in phosphate-buffered saline at PH 7.4 for 15 min at room temperature. The unreacted sodium periodates were neutralized by serine and removed by a desalting column. Finally alkoxyamine-derivatized peptide agents (e.g., NDP-MSH) were synthesized and conjugated to the anti-PD-L1 heavy chain by the oxime ligation in phosphate-buffered saline PH 6 with a catalyst p-anisidine for 3 h at room temperature. The product was purified by size-exclusion chromatography to remove excess non-conjugated NDP-MSH peptide. As shown in FIG. 5, the antibody drug conjugates (ADC) were examined with LC-MS, and as expected the drug-to-antibody ratio (DAR) is 2.

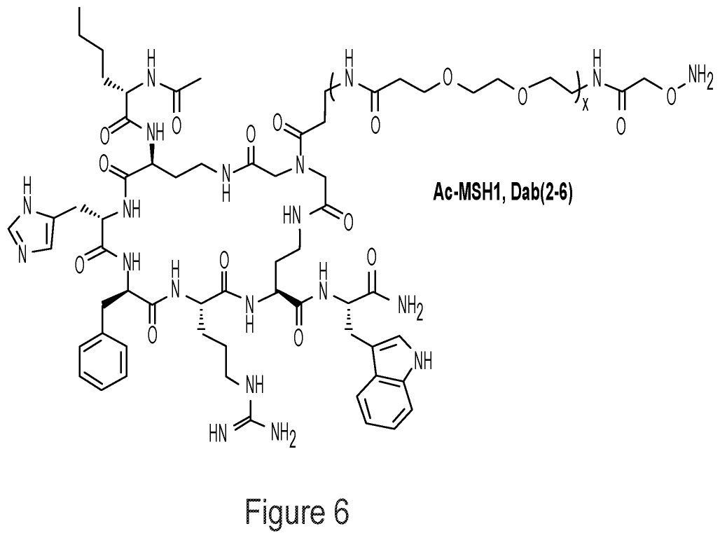

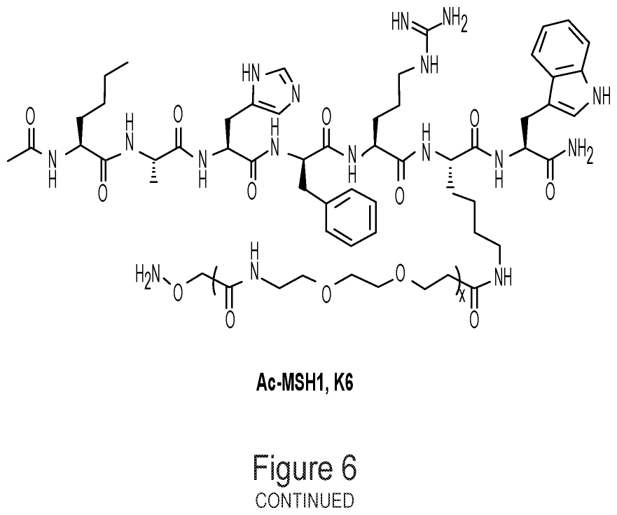

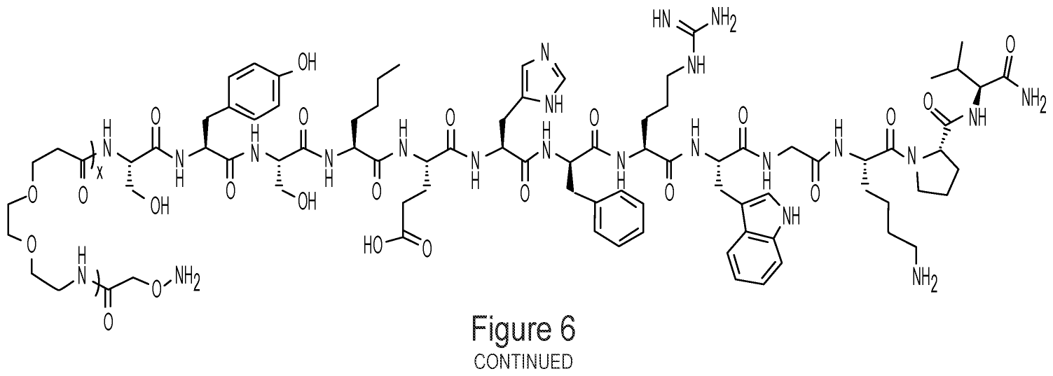

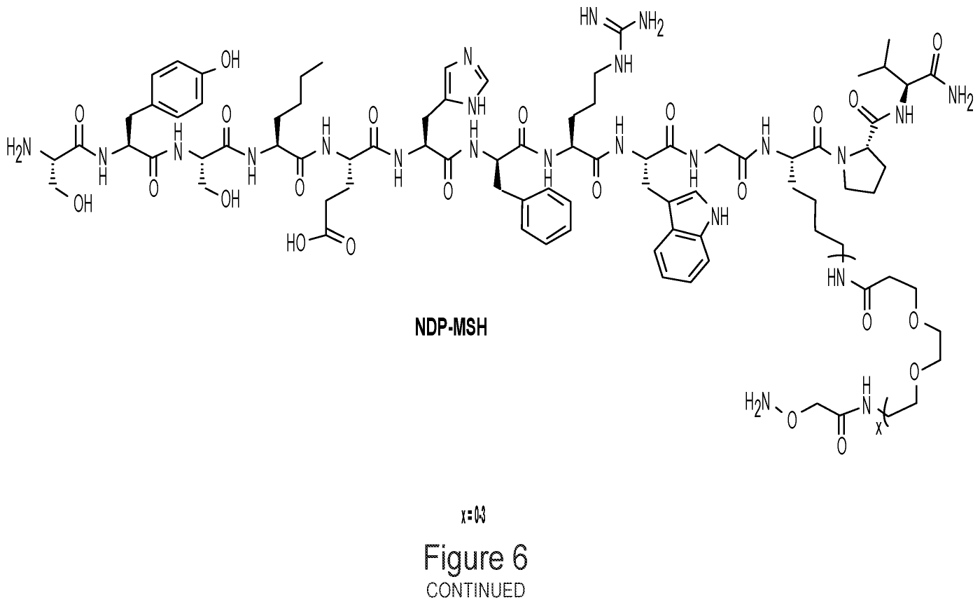

We then optimized the ADC construct with MSH analogs for the highest binding affinity to MC1R. α-Melanocyte stimulating hormone (α-MSH), a tridecapeptide, is the natural ligand with nanomolar binding affinity to MC-1R. We synthesized series of α-MSH analogs with different linkers and conjugation sites. Some of the compounds are shown in FIG. 6. We conjugated the MSH analogs with different linkers to control antibody Synagis via the N-terminus serine conjugation, and analyzed the conjugates via LC-MS. The results indicated the conjugation reaction is completed and the conjugation efficiency is more than 90%.

We characterized the various peptides and antibody conjugates by an in vitro MC1R cAMP activity assay. The B16-SIY cell, which has surface MC1R that can be activated by MSH, was used. The downstream cAMP signaling can be induced and read by the cAMP-Glo™ Assay. Specifically, B16-SIY cells were grown in DMEM with 10% FBS and 1% penicillin and streptomycin. Cells were seeded in 384-well plates at a density of 5000 cells per well and treated with various concentrations of peptides, Synagis conjugates or anti-PD-L1 conjugates for 24 hours at 37° C. with 5% CO2. Luminescence intensities were then measured using cAMP-Glo (Promega, Wis.) following manufacturer's instruction. Data were plotted and analyzed in Graphpad Prizm by non-linear regression in the model of log (inhibitor) vs. response. Based on the results obtained from the assays, we chose H2NO-NDP-MSH as our targeting component and conjugated it on both Synagis and anti-PD-L1. Other than the 8×G4S linker, these conjugates do not contain other linker moiety between the peptide analog and the N-terminus of the antibody.

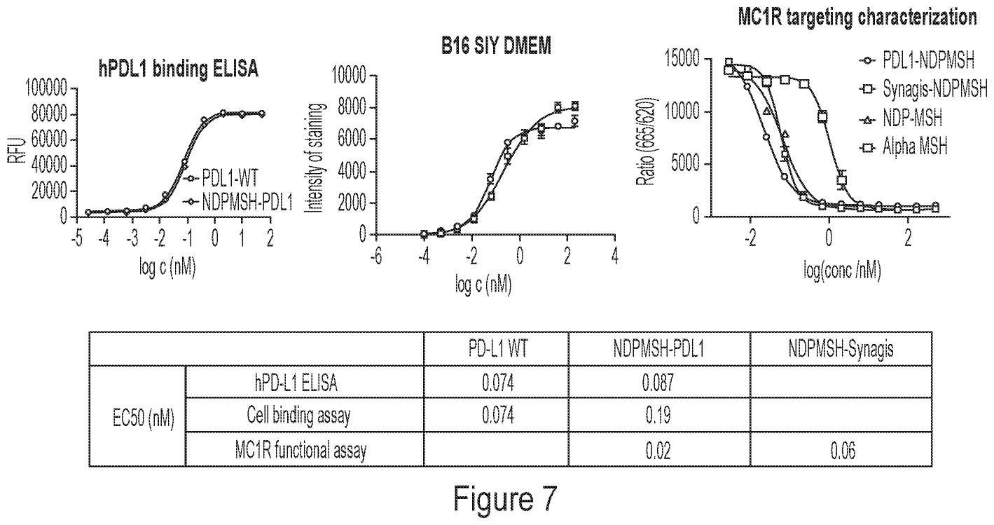

The conjugates were examined for binding to PD-L1 and MC1R. The results are shown in FIG. 7. Specifically, NDP-MSH-αPD-L1 shows nearly the same binding affinity (EC50=0.087 nM) to a human PD-L1 (extracellular domain)-Fc fusion protein by ELISA as that of αPD-L1 antibody alone (EC50=0.074 nM). This result indicates that this conjugation site does not significantly affect binding of the conjugated antibody to PD-L1. The potency of the NDP-MSH-αPD-L1 to the human MC1R was analyzed by the B16-SIY cells cAMP-Glo™ Assay. Both NDP-MSH-Synagis and NDP-MSH-anti-PD-L1 show similar potency with NDPMSH peptide along, which indicates NDP-MSH-αPD-L1 and NDP-MSH-Synagis can activate MC1R with nanomolar potency (EC50=0.02 nM and 0.06 nM respectively), similar to the NDP-MSH (EC50=0.06 nM), in this cell-based activation assay. Based on this result we chose NDPMSH-Synagis and anti-PD-L1 as our controls for the in vivo study.

### Example 6. In Vivo Activities of N-Terminal Conjugated αPD-L1 Bispecific Molecules

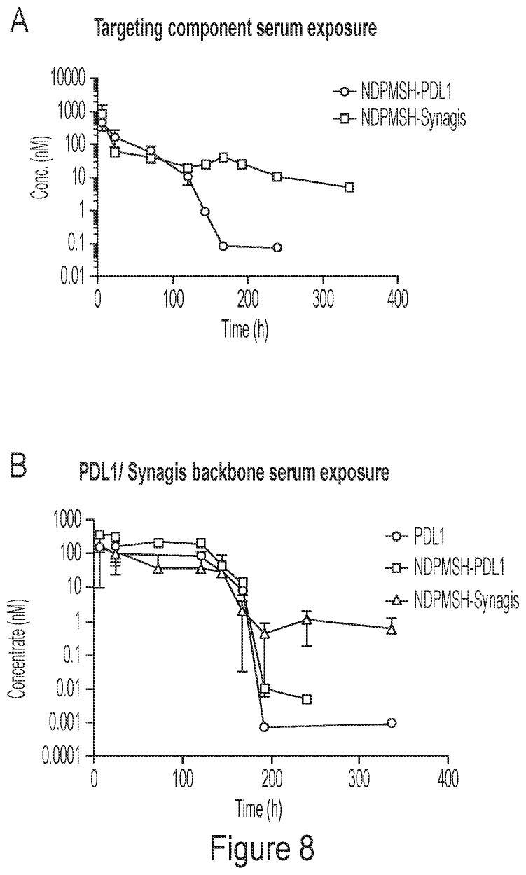

We carried out a pharmacokinetic (PK) study in mice to determine the serum half-life and exposure of NDP-MSH-αPD-L1. Single doses (4 mg/kg) of NDP-MSH-αPD-L1, NDP-MSH-Synagis and αPD-L1 in PBS (pH 7.4) was dosed I.P. to C57/B6 mice (n=6) and plasma samples collected at 7, 24, 72, 144, 168, 192, 240 and 336 h via retro-orbital bleeding. ELISA based assays was used to quantify the plasma concentration of PD-L1 and synagis backbone and MC1R functional assay was used to calculate the NDP-MSH exposure in plasma. As shown in FIG. 8, all of the tested molecules have a high level exposure up to 144 hour and NDP-MSH conjugation is hydrolytic stable and exceeds efficacious concentration (0.02 nM) beyond 144 hour.

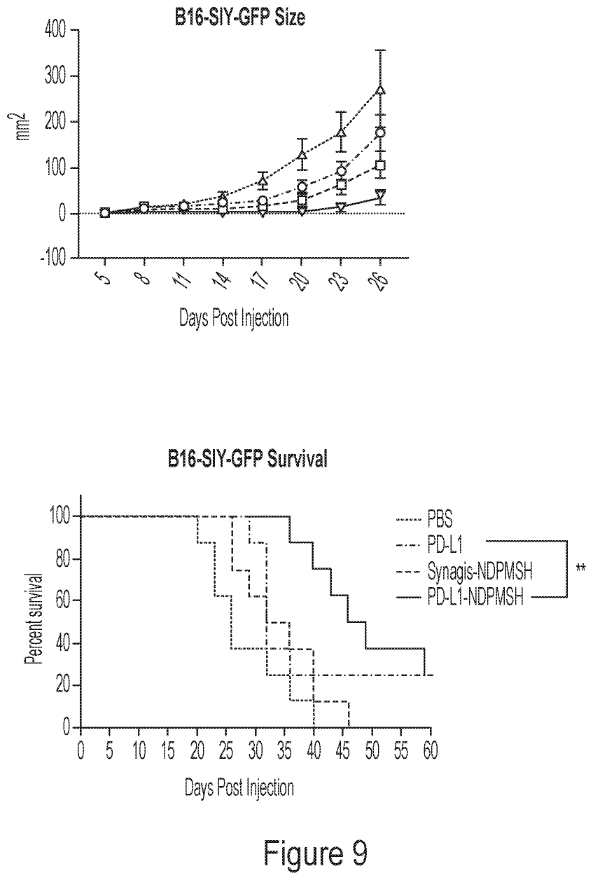

We further examined activities of the conjugates in an in vivo syngeneic model. B16-F10 is a MC1R+/PD-L1+ melanoma cell line that was utilized for the studies of MC1R-targeted radiotherapies. B16-SIY is a derivate cell line from B16-F10 expressing an engineered model antigen SIYRYYGL (SIY), which are more immunogenic than B16-F10 cells and responsive to αPD-L1 treatment murine. A B16-SIY syngeneic mouse model was chosen to compare the in vivo efficacy of αPD-L1, NDP-MSH-Synagis and NDP-MSH-αPD-L1. As shown in FIG. 9, the results indicate that treatment with NDP-MSH-αPD-L1 produced a significant antitumor effect. Mice treated with the 4 mg/kg dose of NDP-MSH-αPD-L1 exhibited a strong tumor growth inhibition. 6 out of 8 mice have no tumor at day 26 and 2 out of 8 mice didn't grow tumor at day 45. The survival curve shows a 2 stars significance compared to the αPD-L1 only treatment group.

### Example 7. Some Exemplified Materials and Methods

Chemicals and peptides. NDP-MSH ([Nle4, D-Phe7]-MSH) with PEG linker (Azido-PEG24-SYS-Nle-EHfRWGKPV-CONH2, Nle=Norleucine, f=D-form Phe) and NR-MSH with peptide PEG linker (NR, Azido-PEG24-SEGYHKSfRP-Nle-WV-CONH2) was synthetized by Innopep Inc. (1R,8S,9s)-Bicyclo[6.1.0]non-4-yn-9-ylmethyl N-succinimidyl carbonate (BCN-NHS) was purchased from Sigma (Cat #744867).

Cloning of antibody expression vector. The genes encoding the αPD-L1 antibody heavy chain and light chain variable regions were synthesized by IDT (Coralville, Iowa) and amplified by polymerase chain reaction (PCR) using PfuUltra II DNA polymerase (Agilent Technologies, CA). The amplified PCR products were cloned to pFuse-hIgG1-Fc backbone vector (InvivoGen, CA) using Gibson assembly kit (NEB, MA). The sequences of the resulting mammalian expression vectors were confirmed by DNA sequencing.

Antibody expression and purification. The expression vector containing the heavy and light chains of the antibody were co-expressed by transient transfection in FreeStyle 293-F cells (Thermo Fisher Scientific, IL), according to the manufacturer's protocol. After adding plasmid-293fectin mixture, cells in flasks were shaken at 125 rpm in a 5% CO2 environment at 37° C. Culture medium containing secreted proteins was harvested and sterile-filtered after 96 h. Antibodies were purified by Protein A chromatography (Thermo Fisher Scientific, IL) and analyzed by SDS-PAGE gel and ESI-Q-TOF protein MS in the presence and absence of dithiothreitol (DTT).

Generation of antibody-peptide conjugates. NHS-BCN was reacted with primary amine of exposed lysines on the surface of αPD-L1 antibody (1 mg/ml) in slightly alkaline phosphate-buffered saline (PBS) conditions (pH 8.3) for 1 h at room temperature to yield stable amide bonds. The NHS-BCN to αPD-L1 antibody molar ratio was optimized at 40 to achieve the best conjugation yield. The reaction mix was loaded onto a 40K MWCO Spin Desalting Column (Thermofisher, Cat #87766) to separate the BCN-conjugated αPD-L1 antibody from free NHS-BCN. The BCN-conjugated αPD-L1 antibody (0.8 mg/ml) was then mixed with Azido-PEG24-NDP-MSH (or —NR) in 1:20 molar ratio. This reaction was carried on in PBS (pH 7.0) and 37° C. for 24 h during which the BCN moiety was covalently ligated with the azido group on the peptide by copper-free click chemistry with a conjugation efficiency >90% based on mass spectroscopy analysis.

Purification and characterization of antibody-peptide conjugates. NDP-MSH-αPD-L1 (or NR-) conjugates were purified by FPLC in PBS (pH 7.4) at 0.4 ml/min flow rate with a size-exclusion column (Superdex 200 10/300 GL, GE Healthcare). UV absorbance at 280 nm was plotted versus the elution time or elution volume. The ligand-to-antibody ratio (LAR) was determined by ESI-Q-TOF protein MS.

Measurement of PD-L1 binding affinity of NDP-MSH-αPD-L1. 100 ng/well human PD-L1-Fc fusion (Sino Biological, China) was coated on 96-well ELISA plates in PBS (pH 7.4) overnight at 4° C., followed by blocking with 2% skim milk in PBS (pH 7.4) for 1 hour at 37° C. After washing with 0.05% Tween-20 in PBS, varied concentrations of NDP-MSH-αPD-L1/NR-αPD-L1/αPD-L1 were added, and incubated for 2 hours at room temperature. 1:2000 diluted HRP-labeled polyclonal anti-human kappa light chain antibody (Thermo Fisher Scientific, IL) was then added and incubated for 2 hours at room temperature. After washing, fluorescence was developed with QuantaBlu fluorogenic peroxidase substrate (Thermo Fisher Scientific, IL), and quantified using a Spectramax fluorescence plate reader with excitation at 325 nm and emission at 420 nm. Data were plotted and analyzed in Graphpad Prizm by non-linear regression in the model of log (agonist) vs. response.

Measurement of MC1R binding affinity of NDP-MSH-αPD-L1. HEK293 cells were grown in DMEM with 10% FBS and 1% penicillin and streptomycin. Cells were transfected with plasmid containing the human MC1R gene using lipofectamine (Life Technologies, MD). The permanently transfected clonal cell line was selected by resistance to G418. MC1R overexpressed cells were cultured on a flat-bottom 96-well plate (black) over night to allow for attachment (2×10⁴/well). After washing with PBS buffer, cells were fixed onto the bottom of wells by spinning down and incubating in 8% paraformaldehyde for 15 minutes. Varied concentrations of NDP-MSH-αPD-L1 or αPD-L1 were added for binding assays. For competition assays, 30 nM of NDP-MSH-αPD-L1 or αPD-L1 in the presence of various concentrations of MSH was incubated with HEK293 MC1R cells. The other ELISA procedures were the same as those published by Abcam (ICE, ab111542). For the final steps, HRP-labeled antihuman IgG (Fc) antibody (ELITechGroup, Netherlands) was diluted 1:1000 in blocking buffer (PBS/5% BSA/0.1% Tween-20), and applied for 1 h followed by extensive washing. QuantaBlu fluorogenic peroxidase substrate was then added and fluorescence signals were obtained as mentioned above.

In Vitro MC1R Activation Assay. HEK 293 cells overexpressing MC1R receptor and CRE-Luc reporter were grown in DMEM with 10% FBS at 37° C. with 5% CO2. Cells were seeded in 384-well plates at a density of 5000 cells per well and treated with various concentrations of conjugates or controls for 24 hours at 37° C. with 5% CO2. Luminescence intensities were then measured using One-Glo (Promega, Wis.) following manufacturer's instruction. Data were plotted and analyzed in Graphpad Prizm by non-linear regression in the model of log (agonist) vs. response.

Flow cytometry analysis of binding to B16-SIY cell line. B16-SIY cells were grown in DMEM with 10% FBS and 1% penicillin and streptomycin. Before analysis, cells were washed with cold PBS (pH7.4) three times, blocked with 2% BSA in PBS, and incubated with 500 nM antibody for 1 hour at 4° C. After removing unbound antibody by washing with 2% BSA in PBS, cells were re-suspended with FITC anti-human IgG Fc (KPL, MD) for 1 hour at 4° C. with gentle mixing, followed by washing with 2% FBS in PBS and analyzed by LSR II flow cytometer equipped (Becton Dickinson, NJ). All results were processed with FlowJo software (TreeStar, OR).

In vivo efficacy study of NDP-MSH-αPD-L1 conjugates. The efficacy study was conducted with 6-week-old female C57BL/6 mice (Jackson Laboratory, n=10). B16-SIY cells were engineered from melanoma cell line B16F10 a model antigen SIYRYYGL (SIY) which can be recognized by CD8⁺ T cells. A total of 1.5×10⁶B16-SIY melanoma cells were injected subcutaneously (s.c.) in the flank of each C57BL/6 mice on Day 0. On day 5 post tumor inoculation, animals were sorted based on tumor volume, and each mouse was dosed intraperitoneally (i.p.) with antibodies or saline for 4 doses, spaced 3 days apart (Day 5, Day 8, Day 11, and Day 14), at 1 mg/kg or 5 mg/kg. Tumors were measured and recorded three times a week with calipers. Tumor volume was calculated based on length×½ (width). Mice were euthanatized at day 23 after tumor injection. Tumors were harvested for further analysis.

Analysis of tumor infiltrated T lymphocytes. Tumor cell suspensions were prepared from solid tumors by enzymatic digestion in HBSS (Thermo Fisher Scientific, IL) containing 1 mg/ml collagenase, 0.1 mg/ml DNase I, and 2.5 U/ml of hyaluronidase with constant stirring for 2 hours at room temperature. The resulting suspension was passed through a 70-um cell strainer, washed once with HBSS and re-suspended in PBS with 3% BSA to a concentration of 1×10⁶ cells/ml for flow cytometric analysis. The frequency of CD3⁺ T cells was determined by staining FITC-labeled anti-mouse CD3 antibody (eBioscience, San Diego, Calif.). Cells were acquired using a LSR II flow cytometer (Becton Dickinson, NJ) and analyzed with FlowJo software (TreeStar, OR). An unpaired t test (two-tailed) was used to compare between two treatment groups. All statistical evaluations of data were performed using Graph Pad Prism software. Statistical significance was achieved at p value <0.05.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications, databases, GenBank sequences, patents, and patent applications cited in this specification are herein incorporated by reference as if each was specifically and individually indicated to be incorporated by reference.

We claim:

1. A bispecific molecule comprising a PD-L1 antibody or antigen-binding fragment thereof, and at least one peptide agent, wherein the peptide agent specifically binds to an antigen or molecular marker on the surface of a tumor cell.

2. The bispecific molecule of claim 1, wherein the PD-L1 antibody is monoclonal antibody Avelumab, Atezolizumab or Durvalumab.

3. The bispecific molecule of claim 1, wherein the tumor cell is a melanoma cell, a breast cancer cell, a lung cancer cell, a kidney cancer cell, an esophageal cancer cell, a gastrointestinal cancer cell or a pancreatic cancer call.

4. The bispecific molecule of claim 1, wherein the tumor cell is a melanoma cell, and the peptide agent specifically binds to MC1R.

5. The bispecific molecule of claim 4, wherein the peptide agent is α-MSH an analog thereof, or a variant thereof.

6. The bispecific molecule of claim 5, wherein the peptide agent is NDP-MSH or a conservatively modified variant thereof.

7. The bispecific molecule of claim 1, wherein the PD-L1 antibody or antigen-binding fragment thereof is covalently fused to the peptide agent.

8. The bispecific molecule of claim 7, wherein the peptide agent is fused to the constant region or the N-terminus of the heavy chain of the antibody.

9. The bispecific molecule of claim 7, wherein the peptide agent is fused to the antibody or antigen-binding fragment thereof via a linker.

10. The bispecific molecule of claim 9, wherein the linker is a PEG linker or a peptide linker.

11. The bispecific molecule of claim 9, wherein each antibody molecule is fused to about 1 to 10 molecules of the peptide agent.

12. The bispecific molecule of claim 1, comprising PD-L1 antibody Avelumab that is fused at the heavy chain constant region to a MC1R-binding peptide agent.

13. The bispecific molecule of claim 12, comprising NDP-MSH or a conservatively modified variant thereof that is covalently fused to the antibody via a PEG linker.

14. The bispecific molecule of claim 1, comprising PD-L1 antibody Atezolizumab that is fused at the heavy chain N-terminus to a MC1R-binding peptide agent.

15. The bispecific molecule of claim 14, comprising PD-L1 antibody Atezolizumab with an engineered N-terminal serine residue for conjugation to the MC1R-binding peptide agent.

16. The bispecific molecule of claim 14, comprising NDP-MSH or a conservatively modified variant thereof that is covalently fused to an engineered N-terminal serine residue on the antibody via a peptide liner.

17. A pharmaceutical composition comprising (1) a therapeutically effective amount of the bispecific molecule of claim 1, and (2) a pharmaceutically acceptable carrier.

18. A method of treating a solid tumor in a subject, comprising administering to the subject a pharmaceutical composition comprising a bispecific molecule, wherein the bispecific molecule comprises a PD-L1 antibody or antigen-binding fragment thereof and a peptide agent that specifically binds to a cell surface antigen or molecular marker of the solid tumor.

19. The method of claim 18, wherein the PD-L1 antibody is monoclonal antibody Avelumab, Atezolizumab or Durvalumab.

20. The method of claim 18, wherein the solid tumor is melanoma.

21. The method of claim 20, wherein the cell surface antigen is MC1R, and the peptide agent is α-MSH, an analog thereof or a variant thereof.

22. The method of claim 21, wherein the peptide agent is NDP-MSH or a conservatively modified variant thereof.

23. The method of claim 18, wherein the peptide agent is covalently linked to a heavy chain constant region or a heavy chain N-terminus of the antibody via a linker sequence.

24. The method of claim 23, wherein the linker is a PEG linker or a peptide linker.
